(19) United States
(12) Patent Application Publication
(10) Pub. No.: US 2006/0281975 A1
(43) Pub. Date: Dec. 14, 2006

(54) Home health care interacting instrument

(76) Inventor: Chang-Ming Yang, Miaoli (TW)

Correspondence Address: HDSL, 4331 STEVENS BATTLE LANE, FAIRFAX, VA, 22033, US

(21) Appl. No.: 11/149,391

(22) Filed: Jun. 10, 2005

(51) Int. Cl. A61B 5/00; A61B 5/02; A61B 5/08

(52) U.S. Cl. 600/300; 600/549; 600/500; 600/529

(57) ABSTRACT

The integration of physical monitors (e.g. electronic stethoscope, sphygmomanometer etc.) with the earphone or microphone and the usage of communications equipments (e.g. cellular, PDA or computer etc.) let the medical personals evaluate the patient's condition immediately on line; the patient can make use of various physical monitors to check himself/herself by following the guidance and instruction from medical personals. In this way, medical personals remotely could get the most correct information and find the real cause of the disease in a short time.

### BACKGROUND OF THE INVENTION

1. Field of the Invention

This invention is a home health care interacting instrument, and a medical tool using communications equipment in particular.

2. Description of the Related Art

Home health care is an important issue to modern life. Its major problems are:
The population structure is aging, having fewer kids and there is increasing demand for care presently.
People are overworking, having more stress, and having no time to go to hospital for checking health condition.
The change of diet and living style results in the origin of various chronic diseases tending to happen in young age.
There is demand for caring but no good assistant tool to provide simple and efficient caring service.
The caring service is too expensive to serve most people in need.

Therefore, home health care should be done in daily life rather than go to hospital until the patient's condition is becoming worse.

Basically, there are several questions that a doctor will ask for diagnose when one sees a doctor (e.g. If patient has fever or ache, or the doctor uses stethoscope to check heartbeat and so on). Checking if there is fever, how is the body temperature, and whether the heartbeat is normal and so on - these are the information important for physician to diagnose.

Medical instruments are important tools for physicians and patients because proper tools not only help physician checking how severe the patient is at that moment but also find the potential reason causing the disease. For example, when using stethoscope to check heartbeat, once the cold stethoscope touches skin making patient shiver. Besides, physician cannot avoid touching patient's body while using stethoscope and that might cause misunderstanding and medical dispute.

There is no doubt that medical instruments are important to physicians and patients. At present, patients need to go to hospital for seeing doctors; if patients are having severe condition, disability or living remotely, additional resources need to be used which is a waste and might not be able to save patients in golden period. This is a vast damage to patients.

Therefore, it is important issues to invent and manufacture a tool that can let patients instantly get professional suggestions and evaluations from medical personals.

### SUMMARY OF THE INVENTION

Based on the defects found in used home health care instrument, the inventor takes time to research and design for improvement and resolution, finally to contrive the means of this interacting home health care instrument.

This invention is to provide an interacting home health care instrument which will enable home health care computerized and efficiency, let everyone easily accesses health care instruction or related information, and continuously measure physical information in a long run. Once the long term and reliable figures is obtained, the medical institute could elevate the quality and result of home health care. This will not only save medical resources from wasting but also improve trust and interaction between physicians and patients.

This invention not only could be used in home health care, but also could be used in severe or distant patient while getting the professional suggestion and evaluation from physician immediately, and then the physician decides whether the patient should go to hospital for further examination. Patients can push the emergency speed button that makes the high decibel sound, lights up and dials to inform the medical personals stay around and distantly to deal with the situation immediately.

To hospital, this saves it from resource wasting. To physicians, this lets them give professional suggestion, evaluation and provide best health care to patients right away. To patients, this would make it possible to get the rescue without delay and get the professional instructions and suggestion, which then reduce the damage to patient.

This invention links to communications equipments, and could utilize some functions (e.g. photographing, video taping, taping and so on) on communications equipments to inform medical personals (e.g. sending the position information of electronic stethoscope through the cellular phone with digital photographing function to medical personals for making sure whether the patient put it on correct place, or transmitting the current image of the patient to medical personals through computer ... and so on). Medical personals could use the basic functionality of these communications equipments to instruct the patients more correctly.

Take electronic stethoscope (physical monitor) as an example, the current body temperature of the user could be taken by an earphone which a body temperature sensor is installed inside; the output terminal of the electronic stethoscope can plug in to the socket of output terminal on the microphone, then user could put the electronic stethoscope on proper position of his/her body by following the physician's instruction and let the physician check whether there is any abnormality of the user's heartbeat and lung sound in order to give professional suggestions and evaluations. In this way, patients can examine by themselves, and the medical dispute of touching body could be avoided.

A thermostat could be installed in the electronic stethoscope to prevent patients from feeling cold once it is put on them. This instrument could be used on animals or machines.

### BRIEF DESCRIPTION OF THE DRAWINGS

The drawings disclose an illustrative embodiment of the present invention that serves to exemplify the various advantages and objects hereof, and are as follows:

FIG. 1 is the first example of the realization of this invention.

FIG. 2 is the second example of the realization of this invention.

FIG. 3 is the third example of the realization of this invention.

FIG. 4 is the fourth example of the realization of this invention.

FIG. 5 is the fifth example of the realization of this invention.

FIG. 6 is the flow chart of this invention.

FIG. 7 is the flow chart of the invention using the analysis software.

### DETAILED DESCRIPTION OF THE PREFERRED EMBODIMENT

To let the examiners further understand the structure, utilization and characteristics of the invention, the inventor presents a better embodiment with figures and detailed description as follows:

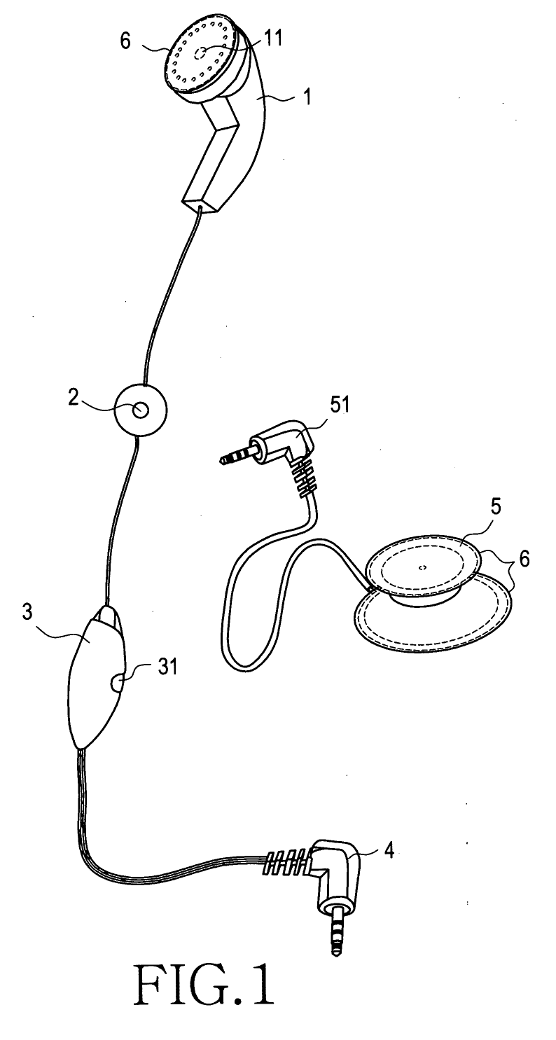

FIG. 1 is taking the electronic stethoscope as an example. A body temperature sensor 11 is installed on the earphone 1 to detect current body temperature of the user. A disposable protecting membrane 6 is attached to the earphone 1 to prevent next user from cross infection. User could plug in the terminal plug 51 of electronic stethoscope 5 into the terminal socket 31 of microphone 3. Plugging in the first terminal plug 4 into communications equipments and using the communications equipments to contact medical personals remotely to accomplish the interacting purpose of this invention; or through connecting electronic stethoscope 5 to communications equipments, medical personals remotely can monitor patients heartbeat and lung sound. To the electronic stethoscope 5 a disposable protecting membrane 6 is attached to prevent cross infection and reduce the possibility of diseases.

The physical monitor is a medical tool that could automatically proceed some medical testing or follow instructions of medical personals. It can save the values of current body temperature, the sound of heartbeat or lung, then make use of some functionalities (e.g. photographing, video taping, taping and so on) of the communications equipments (e.g. cellular phone, PDA, computer and so on) sending the information to medical personals as references. This instrument not only serves as a medical tool daily, but also serves as a speakerphone. This invention makes home health care possible and prevents patient's condition from becoming severe.

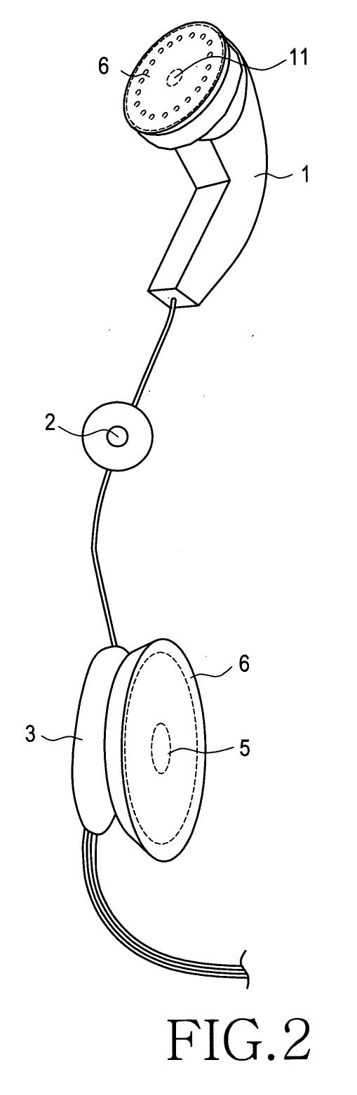

FIG. 2 is another implementation of this invention. The electronic stethoscope 5 without the terminal plug 51 is integrated into the microphone 3 like conjoint that reduces the situation of mal-connecting or plug detaching.

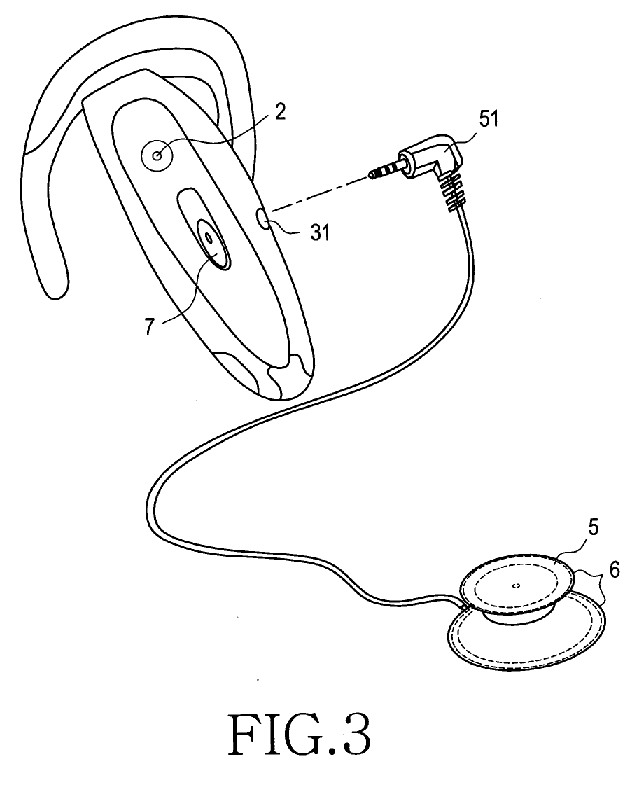

FIG. 3 illustrates the integration of bluetooth technology and the electronic stethoscope. Plugging in the terminal plug 51 of electronic stethoscope into the terminal socket 31 of cellular with bluetooth technology lets medical personals distantly monitor heartbeat and lung sound of patients. A disposal protecting membrane is attached to the electronic stethoscope to prevent cross infection. This instrument could automatically take current temperature of the patient, then save as a record and transmit the information by using bluetooth technology to cellular, PDA or computer and so on. In this way, the invention could break the limitation of distance, let physician diagnose and give instruction precisely base on the basic information under any situation. This instrument could serve as a medical tool; it could also serve as an earphone with bluetooth technology. User can turn it off by switch 7 to save power.

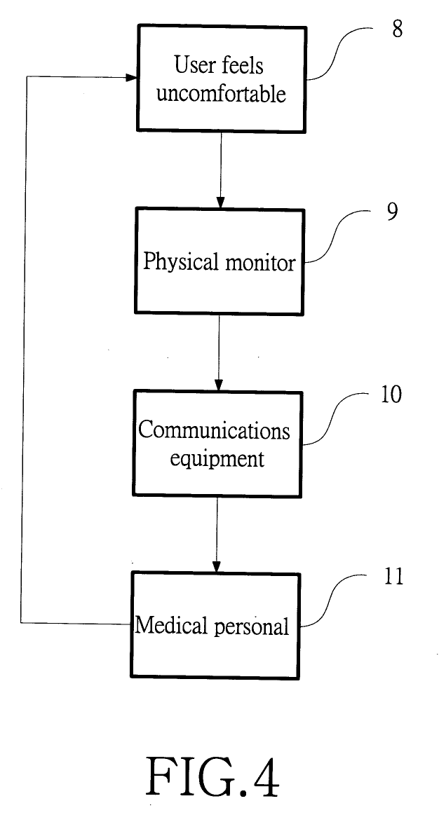

The fourth implementation of this invention is illustrated in FIG. 4. The invention is installed inside the communications equipment (e.g. cellular phone, PDA etc.), and there is a physical monitor 100 and a removable mouthpiece 101 on the flat side of the communications equipment. When user feels uncomfortable, he/she can use this communications equipment to monitor his/her current health status. The device is put on different part of user's body depending on the type of physical monitor inside the communications equipment used. When using the lung function monitor installed on the communications equipment, user breathes into the removable mouthpiece 101 to check lung fimction directly. The mouthpiece could be removed from the device for easily carrying while not used.

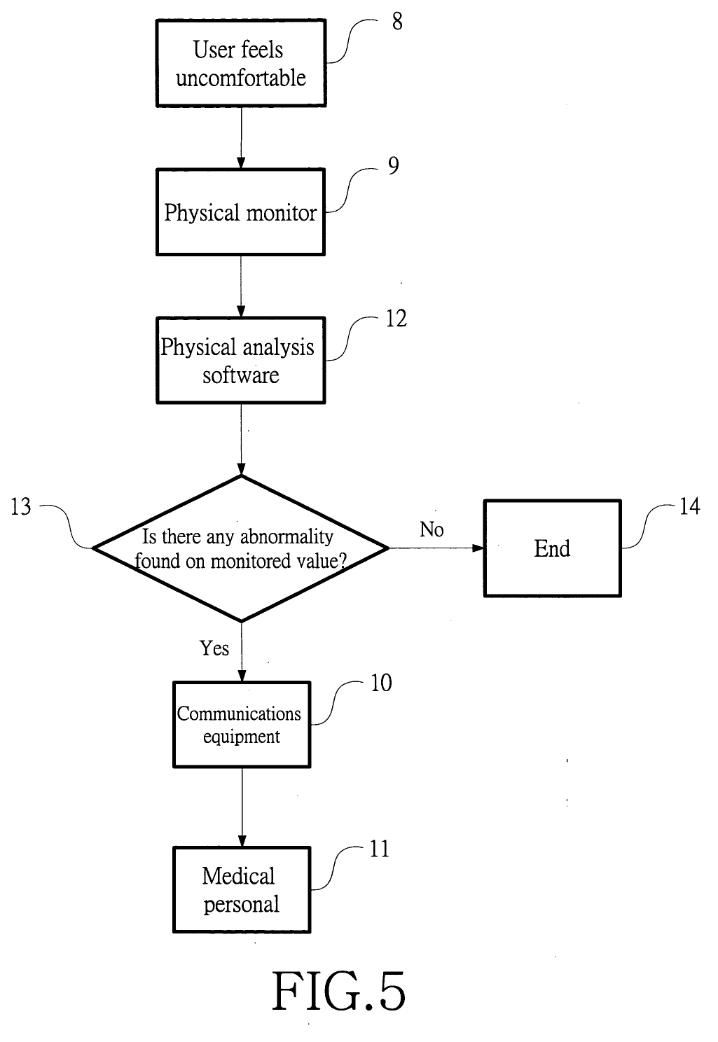

FIG. 5 illustrates the fifth implementation of this invention using the RFID technology. An RFID reader 120 is installed in the communications equipment (e.g. cellular phone or PDA and so on). User wears a wireless physical monitor 110 which includes a physical monitor 100 and an RFID label 111, the physical monitor 110 enables whoever wears it on to check different physical status by using different physical monitor, and to receive various values measured.

As illustrated in FIG. 6, when user feel uncomfortable 200, he/she can put the physical monitor 100 at proper position; then the medical personals 202 remotely can diagnose by using the basic functionalities (e.g. photographing, video taping, taping etc.) of communications equipments 201 (e.g. cellular, PDA or computer etc.) and use these functionalities to check patient's current condition in order to give professional suggestion.

FIG. 7 is the second example implementation of this invention. While feel uncomfortable 200, the user puts the physical monitor 100 on proper position, through the analysis of physical analysis software 203 to judge whether the monitored value is normal 204, if nothing wrong then the process is terminated 205. If abnormality were found, the physical analysis software would inform the medical personals 202 remotely through the communications equipments 201.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. An interacting home health care instrument, comprising:
a communications equipment;
an earphone;
a microphone;
a physical monitor;
the earphone, the microphone and the physical monitor linking to the communications equipment to provide two-way conversation between medical personal and user; the interacting home health care instrument being put on users' body for self checking if there is any abnormalities; the communications equipment contacting the medical personal distantly when abnormal condition is found; the earphone and the microphone being used for two-way communication; the medical personals remotely checking patients' condition through the physical monitor.

2. An interacting home health care instrument as recited in claim 1, wherein the communications equipments could be cellular phones, PDAs, radio devices, or instruments connected to internet.

3. An interacting home health care instrument as recited in claim 1, wherein a protecting membrane with hard or soft texture is attached to the earphone.

4. An interacting home health care instrument as recited in claim 1, wherein the earphone could be with wire or wireless.

5. An interacting home health care instrument as recited in claim 1, wherein a body temperature sensor is installed in the earphone.

6. An interacting home health care instrument as recited in claim 1, wherein a emergency speed button could be set up between the earphone and the microphone for informing medical personals at remote site through communication equipments to provide emergency aids.

7. An interacting home health care instrument as recited in claim 5, wherein a buzzer is installed inside the emergency speed button.

8. An interacting home health care instrument as recited in claim 1, wherein the microphone could be with wire or wireless.

9. An interacting home health care instrument as recited in claim 1, wherein the physical monitor is conjoining with the microphone, and the physical monitor is integrated into the microphone to avoid mal-connecting or terminal plug detaching.

10. An interacting home health care instrument as recited in claim 1, wherein a protecting membrane with hard or soft texture.

11. An interacting home health care instrument as recited in claim 1, wherein a thermostat is installed inside the physical monitor.

12. An interacting home health care instrument as recited in claim 1, wherein the physical monitor could be an electronic stethoscope.

13. An interacting home health care instrument as recited in claim 1, wherein the physical monitor could be a sphygmomanometer.

14. An interacting home health care instrument as recited in claim 1, wherein the physical monitor could be a pulse monitor.

15. An interacting home health care instrument as recited in claim 1, wherein the physical monitor could be a glucometer.

16. An interacting home health care instrument as recited in claim 1, wherein the physical monitor could be a bodyfat monitor.

17. An interacting home health care instrument as recited in claim 1, wherein the physical monitor could be a lung function monitor.

18. An interacting home health care instrument as recited in claim 1, wherein the physical monitor could be an ultrasound system.

19. An interacting home health care instrument as recited in claim 1, wherein the physical monitor could be a blood gas monitor.

20. An interacting home health care instrument as recited in claim 1, wherein the physical monitor could be an electrocardiography instrument.

21. An interacting home health care instrument as recited in claim 1, wherein the physical monitor could be a heart rate variability (HRV) monitor.

22. An interacting home health care instrument as recited in claim 1, wherein the physical monitor could be installed inside the communications equipment and user could use the communications equipment for checking purpose directly.

23. An interacting home health care instrument as recited in claim 1, wherein a thermostat could be installed inside communications equipment to prevent user from feeling cold while using the device.

24. An interacting home health care instrument as recited in claim 1, wherein a removable mouthpiece is installed on the flat side of the communications equipments for user to check lung function with lung function monitor.

25. A home health care instrument using RFID, comprising:
a communications equipment;
a physical monitor,
an RFID reader;
wherein the RFID label is installed inside the physical monitor for user to monitor current health status with various kinds of physical monitors, and the RFID reader reading the data in RFID label is installed inside communications equipment receiving the value measured.

26. A home health care instrument using RFID as recited in claim 25, wherein the communications equipments could be cellular phones, PDAs, radio devices or instruments connected to internet.

27. A home health care instrument using RFID as recited in claim 25, wherein the physical monitor could be a body temperature sensor.

28. A home health care instrument using RFID as recited in claim 25, wherein the physical monitor could be an electronic stethoscope.

29. A home health care instrument using RFID as recited in claim 25, wherein the physical monitor could be a sphygmomanometer.

30. A home health care instrument using RFID as recited in claim 25, wherein the physical monitor could be a pulse monitor.

31. A home health care instrument using RFID as recited in claim 25, wherein the physical monitor could be a glucometer.

32. A home health care instrument using RFID as recited in claim 25, wherein the physical monitor could be a bodyfat monitor.

33. A home health care instrument using RFID as recited in claim 25, wherein the physical monitor could be a lung function monitor.

34. A home health care instrument using RFID as recited in claim 25, wherein the physical monitor could be an ultrasound system.

35. A home health care instrument using RFID as recited in claim 25, wherein the physical monitor could be a blood gas monitor.

36. A home health care instrument using RFID as recited in claim 25, wherein the physical monitor could be an electrocardiography instrument.

37. A home health care instrument using RFID as recited in claim 25, wherein the physical monitor could be a HRV monitor.
